(19) United States
(12) Patent Application Publication
(10) Pub. No.: US 2011/0076714 A1
(43) Pub. Date: Mar. 31, 2011

(54) Method for the presence detection of microorganisms in a bioreactor, bioreactor and arrangement

(75) Inventors: Joachim Bangert, Erlangen (DE); Silke Ebbing, Karlsruhe (DE); Peter Maier, Karlsruhe (DE)

(73) Assignee: Siemens AG, Munchen (DE)

(21) Appl. No.: 12/672,456

(22) PCT Filed: Aug. 6, 2007
(86) PCT No.: PCT/EP07/07134

§ 371 Date: May 12, 2010

(51) Int. Cl. C12Q 1/04; C12M 1/34

(52) U.S. Cl. 435/34; 435/287.1

(57) ABSTRACT

The invention relates to a method for the presence detection of microorganisms in a bioreactor (B), to a bioreactor (B), and to an arrangement for performing the detection method. In order to reduce the staffing expense during the detection analysis of the presence of undesirable microorganisms in a bioreactor (B) after the bioreactor (B) has been cleaned, the detection method is carried out for microorganisms present in a bioreactor (B) such that a) a nutrient medium is selectively applied to defined areas of the bioreactor (B) in a controlled manner by a control unit (SE), b) an incubation having a defined time is performed in the bioreactor (B), c) after the incubation has been completed, the nutrient medium is removed from the defined areas in the bioreactor (B) and fed to an analysis unit (AE), in which the presence of microorganisms or decomposition products is determined, d) the analysis result determined in c) is then sent to the control unit (SE), which based on the results of the analysis selects a suitable process step from the predefined process steps for the further operation of the bioreactor (B).

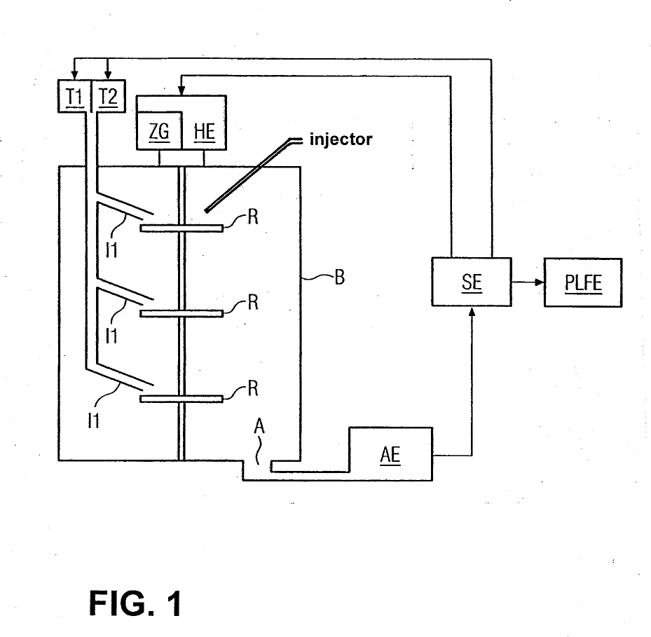

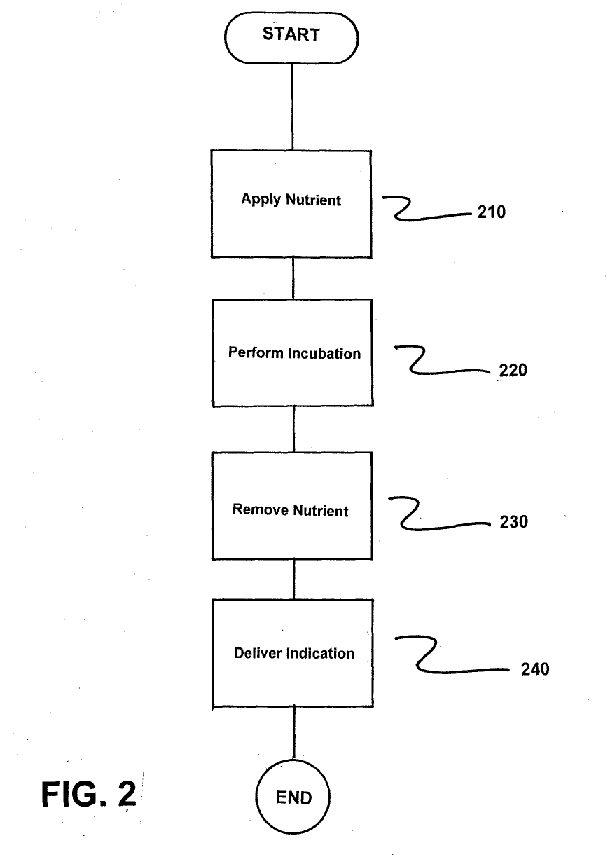

The invention relates to a method for detecting the presence of microorganisms in a bioreactor, to a bioreactor and to an arrangement for carrying out the detection method.

Biological and chemical pharmaceutical preparations, additives for foodstuffs etc. are produced in bioreactors by using biotechnology methods. After a production run, a so-called batch, the bioreactor must be cleaned. After the cleaning has been carried out, it is necessary to detect whether the bioreactor is sterile, i.e. there are no microorganisms in the bioreactor.

Once this verification of the absence of microorganisms has been carried out satisfactorily, the bioreactor is enabled for the next production run.

In respect of the production of pharmaceutical preparations, the cleaning specifications and the specifications for verifying the absence of microorganisms are given by the guidelines for the production of pharmaceutical products, which are essentially established by the US Food and Drug Administration (FDA).

In respect of biotechnological production, it is necessary to ensure that the bioreactor does not contain any other undesired microorganisms in addition to the desired microorganisms. Undesired microorganisms kill the microorganisms to be produced, lead to the creation of undesired byproducts or at least lead to a reduced yield in the production process. Depending on the type of biotechnological production, up to 30% losses can occur in a production run owing to the presence of undesired microorganisms.

The bioreactor is cleaned after a production run by known methods such as cleaning with hot steam, fungicides or similar agents. As already mentioned, it is then necessary to check that the cleaning has been sufficiently effective.

Verification of sufficiently effective cleaning could involve known tests which determine the presence of DNA. These detection methods, however, have proven to be impractical for the task in question here because DNA can in practice always be detected after a production run since the known tests cannot reliably distinguish living DNA from DNA substrands. Even if the cleaning, for example with fungicides, has reliably killed fungal pathogens, the bioreactor would still contain their genetic material or parts thereof which may also be detected. Unreliable cleaning, however, would be critical.

From the internal prior art, it is known to take wipe samples from particular sites in the bioreactor by hand using sterile swabs after having cleaned the bioreactor. The particular sites are generally the bioreactor walls, the feeds and discharges of the bioreactor and further sites which are particularly difficult to access. The individual samples are then smeared onto growth media for microorganisms and incubated at from 30 to 37 degrees Celsius for at least 48 hours. The subsequent evaluation in respect of the presence or non-presence of undesired microorganisms is carried out by visual identification of microorganism colonies which have grown.

It is therefore an object of the present invention to provide a technical solution for detecting the presence of undesired microorganisms in a bioreactor after the bioreactor has been cleaned, which can be done with less work by staff and time expenditure.

The object is achieved by a method for detecting microorganisms contained in a bioreactor, in which

a) a nutrient medium is selectively applied at defined sites in the bioreactor under the control of a control unit,

b) incubation is carried out for a fixed time in the bioreactor,

c) after the incubation has been carried out, the nutrient medium is removed from the defined sites in the bioreactor and sent to an analysis unit, in which the presence of microorganisms or breakdown products is determined,

d) the analysis result determined in c) is subsequently delivered to the control unit, which selects a suitable process step on the basis of the analysis result from among predetermined process steps for further operation of the bioreactor.

The object is furthermore achieved by a bioreactor for carrying out those steps of the detection method as claimed in one of patent claims 1 to 3 which take place in the bioreactor, having an injector for selective application of nutrient medium, which is directed at a defined site in the bioreactor, and an outlet.

The object is furthermore achieved by an arrangement for carrying out the detection method as claimed in one of patent claims 1 to 3, the arrangement comprising the following elements:

a) a bioreactor as claimed in one of patent claims 4 to 6, wherein the injector of the bioreactor is connected to a tank for transferring nutrient medium stored in the tank,

b) an analysis unit connected to the outlet of the bioreactor,

c) a control unit connected to the analysis unit, the tank and a production run enabling unit.

By virtue of the detection method according to the invention, the bioreactor according to the invention and the arrangement according to the invention, after the bioreactor has been cleaned the presence of undesired microorganisms in the bioreactor can be detected with little work by staff. The technical solution furthermore increases the reliability of the detection test since it is not necessary to open the bioreactor and the possible contamination by laboratory staff associated with opening it is avoided. The automation of the technical solution also simplifies validation of the individual process steps and documentation of the detection test.

Refinements of the invention may be found in the dependent claims.

Advantageously, the detection method is refined in that the suitable process step selected by the control unit is repetition of the detection method. Losses in the production run due to the presence of microorganisms still contained in the bioreactor can thereby be avoided.

Also advantageously, the detection method as claimed in patent claim 1 is refined in that the suitable process step selected by the control unit is the start of the normal production run. In this way, the new production run can be initiated in an automated fashion after the absence of microorganisms in the bioreactor has been verified.

Advantageously, the bioreactor is refined in that the injector is used for selective application of the washing medium. A separate injector just for the washing medium can thereby be obviated.

Also advantageously, and as an alternative to the preceding paragraph, the bioreactor is refined in that the bioreactor comprises a separate injector for selective application of the washing medium. This is advantageous in particular when washing medium residues in the injector can lead to contamination of the nutrient medium.

Also advantageously, the arrangement is refined in that the control unit is connected to a heating element which is connected to the bioreactor and is adapted to control the temperature of the incubation in the bioreactor. In this way, an optimally adapted thermal environment can be set up in the bioreactor for different nutrient media.

Other advantages of the invention may be found in the following description, which explains the invention with the aid of an exemplary embodiment in connection with the appended drawing.

In a schematic representation, the

FIGURE shows an arrangement comprising a bioreactor and other components necessary for carrying out the method for detecting the presence of undesired microorganisms in the bioreactor.

The FIGURE shows an arrangement comprising a bioreactor B and other components necessary for carrying out the method for detecting the presence of undesired microorganisms in the bioreactor B. A bioreactor B intended for the production of pharmaceutical or biotechnological products naturally comprises other components, although these are not essential for presentation of the invention and are therefore not shown. In particular, such a bioreactor B comprises separate inlets for substances to be mixed in the bioreactor B during the production run, and outlets for the reaction products formed in the bioreactor B during the production run.

The bioreactor B comprises a stirrer R for accelerating the homogenization of the substances to be mixed. This stirrer R, having three stirring paddles, is driven by an electric motor (not shown here). The injector 11 is directed at the stirring paddles of the stirrer R. The injector 11 is supplied with the nutrient medium stored in the tank T1 and with the washing medium stored in the tank T2.

Connected to the bioreactor B, there is a heating element HE having a timer ZG for time- and temperature-dependent incubation in the bioreactor B.

The bioreactor B comprises an outlet A through which the medium, nutrient medium or washing medium, can reach the analysis unit AE via a tube or hose.

The detection method is controlled by a control unit SE which is connected to the analysis unit AE, the heating element HE, the tanks T1, T2 and a production run enabling unit PLFE.

The detection method will be presented in detail below:

After the process (not represented here) of cleaning the bioreactor B has been completed, nutrient medium from the tank T1 is selectively applied by means of the injector 11 at defined sites on the stirring paddles of the stirrer R.

After appropriate preliminary tests, for example by means of the manual swab sampling and examination known from the internal prior art and described above, the person skilled in the art will be aware of the sites in the bioreactor B where the undesired microorganisms remain. The stirring paddles are merely used for illustration here. Other critical sites where undesired microorganisms predominantly remain are seals, joints, flanges, pores etc. in the bioreactor B. These critical sites are often inaccessible for the described manual detection method. The reliability of the detection method according to the invention can therefore be increased by suitable configuration of an injector which is directed at these critical sites.

The directed selective application of nutrient medium may also be carried out by means of other suitable components (not shown here), such as a sterilized spray head controlled by the control unit SE or by means of a nozzle. A wide variety of possibilities for the selective application of nutrient medium onto the critical sites are known to the person skilled in the art.

The amount and position of the nutrient medium to be applied will be controlled by means of the control unit SE. The nutrient medium to be used will be selected by the person skilled in the art while taking the relevant undesired microorganisms into account.

After the selective application of nutrient, incubation is carried out in the bioreactor B. The incubation time and the temperature in the bioreactor B will likewise be controlled by the control unit SE, and depend inter alia on the type of undesired microorganisms and the nutrient medium selected. For example, infrared lamps or electric heaters are used as heating elements HE.

After the incubation time has elapsed, either more nutrient medium in excess from the tank T1 or a washing solution from the tank T2 is delivered onto the critical sites by the injector 11, by means of which the nutrient medium applied onto the critical sites in the bioreactor B is removed from the critical sites. In an alternative configuration (not shown here), the washing medium is delivered onto the critical sites by means of an injector structurally separate from the injector I1. The rinsing may be carried out by high pressure or pulsed spraying by means of the injector 11 and/or the structurally separate injector. At the end of the rinsing process, the bioreactor may again be washed with a special cleaning or sterilizing solution. The solution removed from the critical sites (nutrient medium optionally mixed with washing, sterilizing and/or cleaning solution) flows through the outlet A and is thus delivered to the analysis unit AE.

The presence of (undesired) microorganisms or breakdown products is then determined in the analysis unit AE. Methods running in an automated fashion, which are known to the person skilled in the art, may be used in the analysis unit AE. As an alternative or in addition, the following analyses may be employed:

Biological analysis, for example filtration and incubation with automatic readout and regeneration of the filter,

Biochemical analysis, for example filtration, cell lysis and detection of specific DNA or cell parts by means of labeled probes,

Chemical analysis, for example of the pH or metabolic products,

Physical analysis, for example fluorescent spectral analysis or automatic microscopy with image evaluation.

The analysis results determined by the analysis unit AE in respect of the presence of (undesired) microorganisms is subsequently delivered to the control unit SE, which then selects a suitable process step on the basis of the analysis result from among predetermined process steps for further operation of the bioreactor B.

One possible selected process step could be to repeat the detection method. This might be necessary if the number of microorganisms determined exceeds a set limit value.

Another possible selected process step could be to start the normal production run. In this case, the control unit SE sends a corresponding signal to the product run enabling unit PLFE.

Another possible selected process step could be to repeat the cleaning of the bioreactor, if the number of microorganisms exceeds a set limit value.

The selection of the suitable process step from among predetermined process steps is carried out by comparison with specifications stored in a memory of the control unit SE. The hardware and software technical implementation required for this is likewise known to the person skilled in the art. The selection of the suitable process step may be based on the change found in the number of microorganisms by a detection method carried out again, the exceeding of limit values, comparison with stored values of microorganisms etc.

The analysis results are electronically saved in a known way and may be documented.

In summary, the detection method according to the invention allows in particular

a) detection of the presence of (undesired) microorganisms at critical sites in the bioreactor B which are difficult to access,

b) an increase in the reliability of the detection method by avoiding contamination of the bioreactor B when opening the bioreactor B, as was previously necessary for the detection test,

c) the practical possibility of carrying out detection tests more frequently owing to the automation of the detection method and the associated lower staff involvement, and comparing their analysis results,

d) documentation of the analysis results simplified by the automation,

e) economical use of the medium owing to the directed application,

f) optimization of the cleaning procedure and the detection method by evaluating the analysis results obtained, which reduces the amount of washing medium and waste water and limits the time taken for the presence detection, and leads to even better selection of the critical sites.

The invention is not restricted to the particular exemplary embodiment but also includes other variants not specifically disclosed, so long as the essence of the invention is used.

1.-8. (canceled)

9. A method for detecting microorganisms contained in a bioreactor, comprising:
applying a nutrient medium selectively at defined sites in the bioreactor under control of a control unit;
performing incubation of the nutrient medium for a fixed time in the bioreactor;
removing, after performing the incubation, the nutrient medium from the defined sites in the bioreactor and sending the nutrient medium to an analysis unit to determine one of an absence and presence of microorganisms or breakdown products in the nutrient medium; and
delivering an indication of one of the absence and presence of the microorganisms or the breakdown products in the nutrient medium to the control unit, the control unit selecting a suitable process step based on the indication of one of the absence and presence of the microorganisms or breakdown products in the nutrient medium from among predetermined process steps for further operation of the bioreactor.

10. The detection method as claimed in claim 9, wherein a suitable process step selected by the control unit is to repeat detection of the presence of one of the microorganisms and the breakdown products in the nutrient medium.

11. The detection method as claimed in claim 9, wherein a suitable process step selected by the control unit is a start of a normal production run.

12. A bioreactor configured to detect microorganisms contained in a bioreactor, comprising:
an injector for selective application of a nutrient medium which is directed at a defined site in the bioreactor; and
an outlet.

13. The bioreactor as claimed in claim 12, wherein the injector is configured to selectively apply a washing medium to the defined sites.

14. The bioreactor as claimed in claim 12, further comprising a separate injector for selective application of the washing medium.

15. An arrangement for performing a detection method in a bioreactor, comprising:
a tank storing a nutrient medium;
a bioreactor having an injector for selective application of the nutrient medium at defined sites in the bioreactor;
an outlet, the injector of the bioreactor being connected to the tank for transferring the nutrient medium stored in the tank through the injector of the bioreactor to the defined sites in the bioreactor;
an analysis unit connected to the outlet of the bioreactor; and
a control unit connected to the analysis unit, the tank and a production run enabling unit.

16. The arrangement as claimed in claim 15, further comprising:
a heating element connected to the control unit and the bioreactor;
wherein the heating element is configured to control a temperature of incubation in the bioreactor.
